(19) United States
(12) Patent Application Publication
(10) Pub. No.: US 2009/0208448 A1
(43) Pub. Date: Aug. 20, 2009

(54) Inhibition of angiogenesis

(76) Inventors: Keith R. Solomon, Boston, MA (US); Kristine Pelton, Abington, MA (US); Carl P. Schaffner, Hamilton, NJ (US); Michael R. Freeman, Boston, MA (US)

Correspondence Address: Cynthia Soumoff;PORZIO, BROMBERG & NEWMAN, P.C., 29 Thanet Road, Suite 201, Princeton, NJ, 08540, US

(21) Appl. No.: 12/378,209

(22) Filed: Feb. 12, 2009

Related U.S. Application Data
(60) Provisional application No. 61/064,088, filed on Feb. 15, 2008.

(51) Int. Cl. A61K 38/20; A61K 31/397

(52) U.S. Cl. 424/85.2; 514/210.02

(57) ABSTRACT

Cholesterol-uptake-blocking drugs inhibit angiogenesis and are useful to inhibit diseases perpetuated by angiogenesis. Cholesterol reduction with the use of the drugs increases the intratumoral level of thrombospondin-1, an angiogenesis inhibitor. Ezetimibe (Zetia®), a specific cholesterol-uptake blocking drug, also retards the growth of human tumors, most preferably in combination with low-cholesterol diet. The pharmacologic reduction in serum cholesterol retards prostate cancer growth by inhibiting tumor angiogenesis to combat the growth of prostatic tumors which are directly accelerated by hypercholesterolemia.

### CROSS REFERENCE TO RELATED APPLICATION

This application claims benefit under 35 U.S.C. § 119(e) of U.S. Provisional Application Ser. No. 61/064,088, filed Feb. 15, 2008, which is herein incorporated in its entirety by reference.

### GOVERNMENT INTERESTS

The invention was made with government support under NIH grants CA101046, NIH R37 DK47556, R01CA112303, and U.S. Army DoD grant PC050337. The government has certain rights in the invention.

### FIELD OF THE INVENTION

The invention pertains to compositions that inhibit angiogenesis and methods of inhibiting diseases by inhibiting angiogenesis.

### BACKGROUND OF THE INVENTION

Angiogenesis is the process by which tissues form new blood vessels from existing ones. This process is essential in many normal processes such as wound healing and muscle growth, but it is also a crucial step in pathological processes such as what occurs during tumor growth and diseases such as macular degeneration. Thus, angiogenesis inhibitors are used to treat a variety of diseases. However, the present drugs have not been entirely successful. Thus, there is an ongoing need for new angiogenesis inhibitors to treat such diseases.

The ongoing research to develop therapies against cancers has implicated high cholesterol levels as having a relationship to the development of this disease. However, the issue of whether circulating cholesterol plays a role in prostate cancer (PCa), or other cancers, is unresolved in the literature. Although, high levels of cholesterol are found in prostate tissue in cases of PCa, they are also found there in normal aging (Freeman, M. R., and Solomon, K. R. 2004. Cholesterol and prostate cancer. J Cell Biochem 91:54-69; Schaffner, C. P. 1981. Prostatic cholesterol metabolism: regulation and alteration. Prog Clin Biol Res 75A:279-324; Swyer, G. I. 1942. The cholesterol content of normal and enlarged prostates. Cancer Res 2:372-375). A number of epidemiological and pre-clinical studies have suggested there is a role for one or more products of the mevalonate/cholesterol synthesis pathway and specifically for high levels of serum cholesterol in PCa incidence and progression (Blais, L., Desgagne, A., and LeLorier, J. 2000. 3-Hydroxy-3-methylglutaryl coenzyme A reductase inhibitors and the risk of cancer: a nested case-control study. Arch Intern Med 160:2363-2368). High fat/high cholesterol ‘Western-type’ diets have been linked to PCa incidence and progression in a number of studies, however a role for specific dietary components in disease progression is disputed (Kolonel, L. N., Nomura, A. M., and Cooney, R. V. 1999. Dietary fat and prostate cancer: current status. J Natl Cancer Inst 91:414-428; Michaud, D. S., Augustsson, K., Rimm, E. B., Stampfer, M. J., Willet, W. C., and Giovannucci, E. 2001. A prospective study on intake of animal products and risk of prostate cancer. Cancer Causes Control 12:557-567). Studies examining groups of nutritional components eaten together suggest that diets with a high content of processed and/or red meat may be associated with higher PCa incidence (Wu, K., Hu, F. B., Willett, W. C., and Giovannucci, E. 2006. Dietary patterns and risk of prostate cancer in U.S. men. Cancer Epidemiol Biomarkers Prev 15:167-171; Walker, M., Aronson, K. J., King, W., Wilson, J. W., Fan, W., Heaton, J. P., MacNeily, A., Nickel, J. C., and Morales, A. 2005. Dietary patterns and risk of prostate cancer in Ontario, Canada. Int J Cancer 116:592-598).

The incidence of prostate cancer has generated a need for reliable treatment. Epidemiological, retrospective and prospective case-control studies of cholesterol-lowering drug use (i.e. HMG-CoA reductase inhibitors, a.k.a. statins) and cancer incidence have shown a negative association between statin use and PCa incidence and/or progression, with some studies showing that longer term statin use leads to reduced risk of advanced disease (Graaf, M. R., Beiderbeck, A. B., Egberts, A. C., Richel, D. J., and Guchelaar, H. J. 2004. The risk of cancer in users of statins. J Clin Oncol 22:2388-2394; Pedersen, T. R., Wilhelmsen, L., Faergeman, O., Strandberg, T. E., Thorgeirsson, G., Troedsson, L., Kristianson, J., Berg, K., Cook, T. J., Haghfelt, T., et al. 2000. Follow-up study of patients randomized in the Scandinavian simvastatin survival study (4S) of cholesterol lowering. Am J Cardiol 86:257-262; Platz, E., Leitzmann, M., Visvanathan, K., and al., e. 2005. Cholesterol-lowering drugs including statins and the risk of prostate cancer in a large prospective cohort study. In Proc Amer Assoc Cancer Res.; Platz, E. A., Leitzmann, M. F., Visvanathan, K., Rimm, E. B., Stampfer, M. J., Willett, W. C., and Giovannucci, E. 2006. Statin drugs and risk of advanced prostate cancer. J Natl Cancer Inst 98:1819-1825). On the other hand, placebo-controlled studies have not supported a link between statins and PCa incidence, with three recent meta-analyses finding no evidence for reduced risk of cancer at any site (including the prostate) in statin-prescribed patient cohorts (Dale, K. M., Coleman, C. I., Henyan, N. N., Kluger, J., and White, C. M. 2006. Statins and cancer risk: a meta-analysis. Jama 295:74-80; Browning, D. R., and Martin, R. M. 2007. Statins and risk of cancer: a systematic review and metaanalysis. Int J Cancer 120:833-843; Baigent, C., Keech, A., Kearney, P. M., Blackwell, L., Buck, G., Pollicino, C., Kirby, A., Sourjina, T., Peto, R., Collins, R., et al. 2005. Efficacy and safety of cholesterol-lowering treatment: prospective meta-analysis of data from 90,056 participants in 14 randomised trials of statins. Lancet 366:1267-1278). Although certain conclusions from these meta-analyses have been challenged (Duncan, R. E., El-Sohemy, A., and Archer, M. C. 2006. Statins and the risk of cancer. Jama 295:2720; author reply 2721-2722; Freeman, M. R., Solomon, K. R., and Moyad, M. 2006. Statins and the risk of cancer. Jama 295:2720-2721; author reply 2721-2722; Salinas, C. A., Agalliu, I., Stanford, J. L., and Lin, D. W. 2006. Statins and the risk of cancer. Jama 295:2721; author reply 2721-2722), they do illustrate that the causal relationship of circulating cholesterol in PCa or other cancers is unresolved.

It is further unresolved because few studies have been designed to directly isolate the role of cholesterol, a neutral lipid critical for cell membranes, from other factors, such as isoprenoids. These lipid moieties modify signaling proteins, such as Ras, Rac and Rho, and are essential for membrane localization. Statins interfere with the mevalonic acid/cholesterol synthesis pathway at an early step, so they also block formation of isoprenoid intermediates upstream from the production of cholesterol. In cell culture and in pre-clinical animal studies, it is apparent that statins affect isoprenylation because bypassing isoprenoid synthesis inhibition reverses statin-induced apoptosis (Boucher, K., Siegel, C. S., Sharma, P., Hauschka, P. V., and Solomon, K. R. 2006. HMG-CoA reductase inhibitors induce apoptosis in pericytes. Microvasc Res 71:91-102). Effects on isoprenoid synthesis have been proposed as the underlying mechanism for the anti-tumor effects of statin drugs (Bassa, B. V., Roh, D. D., Vaziri, N. D., Kirschenbaum, M. A., and Kamanna, V. S. 1999. Effect of inhibition of cholesterol synthetic pathway on the activation of Ras and MAP kinase in mesangial cells. Biochim Biophys Acta 1449:137-149). However, this mechanism is contentious because statins, within standard doses, do not accumulate in peripheral tissues in a concentration sufficient to interfere with isoprenoid synthesis (Desager, J. P., and Horsmans, Y. 1996. Clinical pharmacokinetics of 3-hydroxy-3-methylglutaryl-coenzyme A reductase inhibitors. Clin Pharmacokinet 31:348-371; Sirtori, C. R. 1993. Tissue selectivity of hydroxymethylglutaryl coenzyme A (HMG CoA) reductase inhibitors. Pharmacol Ther 60:431-459; Solomon, K. R., and Freeman, M. R. 2007. Do the cholesterol-lowering properties of statins affect cancer risk? Trends Endo. Met In Press).

Zhuang et al. demonstrated that the atherogenic Paigen diet, which causes hypercholesterolemia, results in more rapid growth of LNCaP human PCa xenografts (Zhuang, L., Kim, J., Adam, R. M., Solomon, K. R., and Freeman, M. R. 2005. Cholesterol targeting alters lipid raft composition and cell survival in prostate cancer cells and xenografts. J Clin Invest 115:959-968). Under the hypercholesterolemic conditions of the diet, tumors accumulated cholesterol in lipid raft membranes, exhibited less apoptosis, and enhanced activation of Akt, a serine-threonine kinase linked to aggressive cancers. Zhuang et al. proposed that cholesterol may be directly contributing to tumor growth by altering signal transduction through effects on lipid rafts (Zhuang, L., Lin, J., Lu, M. L., Solomon, K. R., and Freeman, M. R. 2002. Cholesterol-rich lipid rafts mediate akt-regulated survival in prostate cancer cells. Cancer Res 62:2227-2231).

Thus, there remains a need to determine what causes tumor growth in prostate and other cancers and, once it is known, there remains a need to develop effective therapeutic methods for preventing these diseases.

### SUMMARY OF THE INVENTION

In one aspect, the invention is a method of inhibiting angiogenesis in a subject with an angiogenesis-related pathology comprising administering a therapeutic amount of an azetidinone. In one embodiment, the pathology is selected from the group consisting of macular degeneration, rheumatoid arthritis, psoriasis, diabetes, glaucoma and obesity. In another embodiment, the inhibition of angiogenesis is attained by administering ezetimibe in an amount that is inhibitory for angiogenesis. In a more preferred embodiment, the inhibition of angiogenesis is attained by administering a therapeutic amount of ezetimibe with a therapeutic amount of an angiogenesis inhibitor class of compounds that includes angiostatin, endostatin, TNP-470, thalidomide, aptamer antagonist of VEGF, batimastat, captopril, interleukin 12, lavendustin A, medroxypregesterone acetate, recombinant human platelet factor 4(rPF4), taxol, tecogalan(=SP-PG, DS-4152), thrombospondin, TNP-470 (=AGM-1470) and bevacizumab (Avastin®). In another embodiment, the azetidinone is ezetimibe and the method further comprises maintaining a low fat/low cholesterol diet regimen.

In another aspect, the invention is a method of inhibiting angiogenesis in a solid tumor comprising administering an azetidinone to a subject with a tumor. For example, in various embodiments of the invention, the tumor is located in the prostate, breast, pancreas, liver, brain, lung, kidney, bladder, bone, heart, testis, uterus, ovaries, neck, mouth, nose, eye, head, colon, rectum, stomach, muscle, cartilage, skin or esophagus.

In one embodiment of this aspect of the invention, the azetidinone is ezetimibe, in a therapeutic amount. In another embodiment, an angiogenesis inhibitor compound is coadministered with the azetidinone. In preferred embodiments, the angiogenesis inhibitor compound is selected from the group consisting of angiostatin, endostatin, TNP-470, thalidomide, aptamer antagonist of VEGF, batimastat, captopril, interleukin 12, lavendustin A, medroxypregesterone acetate, recombinant human platelet factor 4 (rPF4), taxol, tecogalan(=SP-PG, DS-4152), thrombospondin, TNP-470 (=AGM-1470) and bevacizumab (Avastin®). In yet another embodiment of this aspect of the invention, the method further comprises maintaining a diet that lowers circulating cholesterol. In one embodiment, the diet comprises maintaining a low fat/low cholesterol diet regimen.

In a related aspect, the invention is a method of suppressing the density of blood vessels in a tumor comprising maintaining a low fat low cholesterol diet and administering a therapeutic amount of ezetimibe.

Another aspect of the invention contemplates a method of elevating the level of thrombospondin-1 at a site exhibiting angiogenesis, comprising administering an azetidinone to a subject with a pathogenic angiogenic condition. In one embodiment of this aspect of the invention, the subject has a tumor at the site.

In another aspect of the invention, what is contemplated is a method of inhibiting tumor cell proliferation comprising administering a therapeutic amount of an azetidinone with a therapeutic amount of an angiogenesis inhibitor to a subject with a solid tumor. For example, in various embodiments the solid tumor site is selected from the group consisting of prostate, breast, pancreas, liver, brain, lung, kidney, bladder, bone, heart, testis, uterus, ovaries, neck, mouth, nose, eye, head, colon, rectum, stomach, muscle, cartilage, skin and esophagus.

In a preferred embodiment, the azetidinone is ezetimibe and the angiogenesis inhibitor is selected from the group consisting of angiostatin, endostatin, TNP-470, thalidomide, aptamer antagonist of VEGF, batimastat, captopril, interleukin 12, lavendustin A, medroxypregesterone acetate, recombinant human platelet factor 4 (rPF4), taxol, tecogalan(=SP-PG, DS-4152), thrombospondin, TNP-470 (=AGM-1470) and bevacizumab (Avastin®). In yet another embodiment of this aspect of the invention, the method further comprises administering a therapeutic amount of an anticancer agent. In preferred embodiments the anticancer agent is selected from the group consisting of a steroidal antiandrogen, a non steroidal antiandrogen, an estrogen, diethylstilbestrol, a conjugated estrogen, a selective estrogen receptor modulator (SERM), a taxane, goserelin acetate (ZOLADEX®), and leuprolide acetate (LUPRON®)

In another aspect, the invention is a method of inhibiting prostate tumor growth without reducing testosterone levels comprising administering a therapeutic amount of an azetidinone. In a preferred embodiment, the azetidinone is ezetimibe. In yet another embodiment, the method further comprises administering a therapeutic amount of an angiogenesis inhibitor. In preferred embodiments of this aspect of the invention, the angiogenesis inhibitor is selected from the group consisting of angiostatin, endostatin, TNP-470, thalidomide, aptamer antagonist of VEGF, batimastat, captopril, interleukin 12, lavendustin A, medroxypregesterone acetate, recombinant human platelet factor 4 (rPF4), taxol, tecogalan (=SP-PG (sulfated polysaccharide-peptidoglycan), DS-4152), thrombospondin, TNP-470 (=AGM-1470)(the fumagillin analog TNP-470) and bevacizumab (Avastin®). In yet a further preferred embodiment, the method further comprises administering a therapeutic amount of a chemotherapeutic agent.

In another aspect, the invention is a method of inhibiting prostate tumor cell proliferation in androgen-suppressed males comprising administering a therapeutic amount of an azetidinone. In a preferred embodiment, the azetidinone is ezetimibe. In a further preferred embodiment, the inhibition is attained by administering a therapeutic amount of ezetimibe with a therapeutic amount of an angiogenesis inhibitor class of compounds that includes angiostatin, endostatin, TNP-470, thalidomide, aptamer antagonist of VEGF, batimastat, captopril, interleukin 12, lavendustin A, medroxypregesterone acetate, recombinant human platelet factor 4(rPF4), taxol, tecogalan(=SP-PG, DS-4152), thrombospondin, TNP-470 (=AGM-1470) and bevacizumab (Avastin®). In another preferred embodiment, the inhibition is attained by administering a therapeutic amount of ezetimibe with a therapeutic amount of a chemotherapeutic agent.

In an example of the various aspects of the invention, the azetidinone is ezetimibe and the method further comprises maintaining a diet regimen that lowers circulating cholesterol.

### BRIEF DESCRIPTION OF THE DRAWINGS

FIG. 1. Diet/ezetimibe effects on serum cholesterol levels and the growth of implanted prostate tumors in SCID mice. (A) Serum cholesterol levels in diet/ezetimibe mouse cohorts. Animals were given various ezetimibe (Z)-diet combinations (see Figure) for 2 weeks after which the mice were bled by small tail vein incision, and cholesterol measured in the collected serum via Infinity colormetric assay. Data are plotted as cholesterol level (mg/dL) vs. group±SE. n=13-15/group. Two-way ANOVA indicated significant effects of diet (F=77.57, p<0.001) and drug (F=23.48, p<0.001) on cholesterol levels, but no significant diet-by-drug interaction (F=0.86, p=0.36) suggesting that the effects are independent. (B) Longitudinal volume measurements. SCID mice were fed various diet/ezetimibe (Z) combinations for 2 weeks prior to tumor implantation (see Materials and Methods). Tumors were measured daily by calipers starting at first appearance (day 1) and continued for 13 days. Data are plotted as tumor volume (mm3) per site vs. Time (days)±SE. A mixed model analysis was used to calculate the significance of the both diet (p=0.048) and ezetimibe (Z) (p=0.035) on tumor growth. n=52-60/group (C) Tumor wet weight. At sacrifice all tumors were removed and weighed. Data are plotted as average tumor mass (g) per site vs. group±SE. These data were statistically significant between LFNC+Z (0.63±0.55 average grams/tumor site) vs. HFHC (0.88±0.71 average grams/tumor site) groups (p=0.021) and between the LFNC (0.67±0.56 average grams/tumor site) vs. HFHC groups (p=0.037). n=52-60/group. In all cases data are considered significant at p<0.05.

FIG. 2. Biochemical and cell biological tumor characteristics. (A) Tumor cholesterol levels. Membranes were prepared from tumors and subjected to cholesterol extraction and analysis (see Materials and Methods). Data are plotted as cholesterol (mg)/(g)ram tumor tissue 22 vs. diet/ezetimibe (Z) group±SE. Data were analyzed by ANOVA, which indicated no significant interaction between diet and ezetimibe (p=0.40) but highly significant main effects of both diet (p=0.039) and ezetimibe (p<0.0001) n=9/group. (B) Tumor cell apoptosis (TUNEL staining). Upper panel: representative TUNEL stained images. Left column shows TUNEL staining (fluorescein; green) of selected tumor sections; middle column shows nuclear counterstaining (DAPI; blue) and right column is the merged image of TUNEL and DAPI counterstaining. n=40. Lower panel: quantitative evaluation of tumor cell apoptosis levels. Data are plotted as relative level of TUNEL staining vs. diet/ezetimibe (Z) group±SE. Data were analyzed by ANOVA, which indicated no significant interaction between diet and ezetimibe (p=0.85) but highly significant main effects of both diet (p<0.0001) and ezetimibe (p<0.0001). (C) Tumor cell proliferation (Ki67 staining). Upper panel: representative Ki67 stained images. Left column, Ki67 staining (red); Right column, merged image of Ki67 (Cy3; red) and DAPI (blue; nuclei) counter-staining. n=20. In (B) & (C) Tumors were fixed in OCT, and 3 μm sections were stained for TUNEL (in B) and for Ki67 (in C). Lower panel: quantitative evaluation of tumor cell proliferation levels. Data are plotted as relative level of Ki67 staining vs. diet/ezetimibe (Z) group±SE. The data were analyzed by two-way ANOVA, which demonstrated that there is a significant ezetimibe by diet interaction (p=0.027), implying that ezetimibe has a significant effect on lowering proliferation although the magnitude of this effect depends on the diet; LFNC: average 28.78 (95% CI; 25.42 - 32.14) w/ezetimibe: average 25.57 (95% CI; 22.21-28.93); HFHC: average 41.16 (CI; 37.80 -44.52) w/ezetimibe: average 30.32 (95% CI; 26.96-33.68). Images were acquired and analyzed by AxioVision 4.0 software for quantification. In all cases data are considered significant at p<0.05.

FIG. 3. Angiogenesis in xenograft tumors. (A) Tumor hemoglobin quantification. Tumors were subjected to mechanical disruption in PBS, followed by centrifugation (to remove debris) and the clarified supernatants analyzed by OD (absorbance at 530 Å—absorbance at 650 Å). Data are plotted as relative hemoglobin/mg tumor tissue vs. group (mean value is indicated by line). All groups demonstrated statistical significance vs. all other groups except for LFNC vs. HFHC+Z (ezetimibe), and LFNC vs. LFNC+Z, which were not statistically different. n=20. (B) Microvessel density (MVD)-CD31 analysis. Upper panel: quantitative evaluation of tumor section CD31 levels. Data are plotted as relative level of CD31 staining vs. diet/ezetimibe (Z) group±SE. Data were analyzed by Mixed Model Analysis, which indicated no significant interaction between diet and ezetimibe (p=0.199) but highly significant main effect of ezetimibe (p=0.013) with larger effects when the HFHC was used (p=0.01). Lower panel: representative anti-CD31 mAb stained images. Left column, CD31 staining (Alexa Fluor 488; Green); Right column, merged image of CD31 (Alexa Fluor 488; Green) and DAPI (blue; nuclei) counter-staining. n=76-103. (C) MVD-caveolin-1 analysis. Immunoblotting. Tumors were subjected to SDEM and raft fractions were then subjected to SDS-PAGE and immunoblot analysis using anti-caveolin-1 mAb. Film exposures were analyzed by densitometry and the signal intensity data normalized for each film so that the largest signal had a value of 1. All signal ratios were then averaged to calculate the relative intensity of caveolin-1 for tumor samples from each diet/drug cohort. Data are plotted as average caveolin-1 signal (arbitrary units) vs. diet/ezetimibe (Z) group±SE. ANOVA analysis indicated no statistically significant diet effect (p=0.171) however a significant drug effect in lowering caveolin for both diet conditions (p=0.027). n=6/group. Immunofluorescence. Upper panel; Quantification of caveolin-1 staining. Data are plotted as relative level of caveolin-1staining vs. diet/ezetimibe (Z) 24 group±SE. Data were analyzed by Mixed Model Analysis, which indicated highly significant main effects of ezetimibe (p<0.0001) and diet (p<0.0001). n=43/group Lower Panel; representative caveolin-1 staining. Sections were stained for caveolin-1 (see Materials and Methods) and nuclei were counter-stained with DAPI. Left column; representative anticaveolin-1 mAb staining (Alexa Fluor 488; green). Right column; representative caveolin-1 staining (Alexa Fluor 488; green) merged with DAPI staining (blue). Tumors were fixed in OCT, and 10 μm sections were stained for the indicated markers. CD31 (PECAM) staining was performed as described46. Fluorescent mages were acquired and analyzed by AxioVision 4.0 software for quantification. In all cases data are considered significant at p<0.05.

FIG. 4. Characterization of the xenograft tumor microenvironment. (A) Fibroblast analysis. Upper panel; Quantification of fibroblast staining. Data are plotted as relative level of fibroblast staining vs. diet/ezetimibe (Z) group±SE. Data were analyzed by Two-way ANOVA analysis, which indicated no statistically significant diet effect, however a statistically significant ezetimibe effect in the HFHC condition was detected (p=0.03). n=55/group. Lower Panel; representative fibroblast staining. Sections were stained using a fibroblast specific mAb (see Materials and Methods) and nuclei were counter-stained with DAPI. Left column; representative anti-fibroblast mAb staining (Alexa Fluor 488; green). Right column; representative fibroblast staining (Alexa Fluor 488; green) merged with DAPI staining (blue). (B) Pericyte coverage (vessel quality). Pericyte coverage of microvessels was determined by smooth muscle actin (SMA) staining of peri-microvesicular regions of stained tumor sections (CD31 staining). Upper panel: quantitative evaluation of tumor section SMA levels. Data are plotted as relative level of SMA staining vs. diet/ezetimibe (Z) group±SE. Data were analyzed by Mixed Model Analysis, 25which indicated no significant interaction between diet and ezetimibe (Z) (p=0.062) but highly significant main effects of ezetimibe (p<0.0001) and diet (p=0.009). Lower panel: representative anti-SMA/anti-CD31 mAb stained images. Columns left to right: SMA staining (Alexa Fluor 568; Red); SMA (Alexa Fluor 568; Red) +CD31 (Alexa Fluor 488; Green) merged images; DAPI (blue), SMA (Alexa Fluor 568; Red), +CD31 (Alexa Fluor 488; Green) merged images. n=34-36. (C). TSP-1 levels in tumors. Thrombospondin-1 (TSP-1) levels in tumor sections were determined by staining tumor sections with anti-TSP-1 and anti-CD31 mAbs. Left panel: quantitative evaluation of tumor section TSP-1 I levels. Data are plotted as relative level of TSP-1 staining vs. diet/ezetimibe (Z) group±SE. Data were analyzed by Mixed Model Analysis, which indicated highly significant main effects of ezetimibe (p<0.0001) and diet (p<0.0001). n=34-39. Right panel; representative anti-TSP-1 (Alexa Fluor 568)/anti-CD31 (Alexa Fluor 488) mAb stained images. Columns top to bottom: DAPI (blue), TSP-1 (Alexa Fluor 568; Red), +CD31 (Alexa Fluor 488; Green) merged images; TSP-1 (Alexa Fluor 568; Red) +CD31 (Alexa Fluor 488; Green) merged images; TSP-1 staining (Alexa Fluor 568; Red); Tumors were fixed in OCT, and either 10 μm sections (part A) or 20 μm sections (parts B+C) were stained for the indicated markers. CD31 (PECAM) staining was performed as described46. Fluorescent mages were acquired and analyzed by AxioVision 4.0 software for quantification. In all cases data are considered significant at p<0.05.

Other features and advantages of the invention will be apparent from the following detailed description, and from the claims.

### DETAILED DESCRIPTION

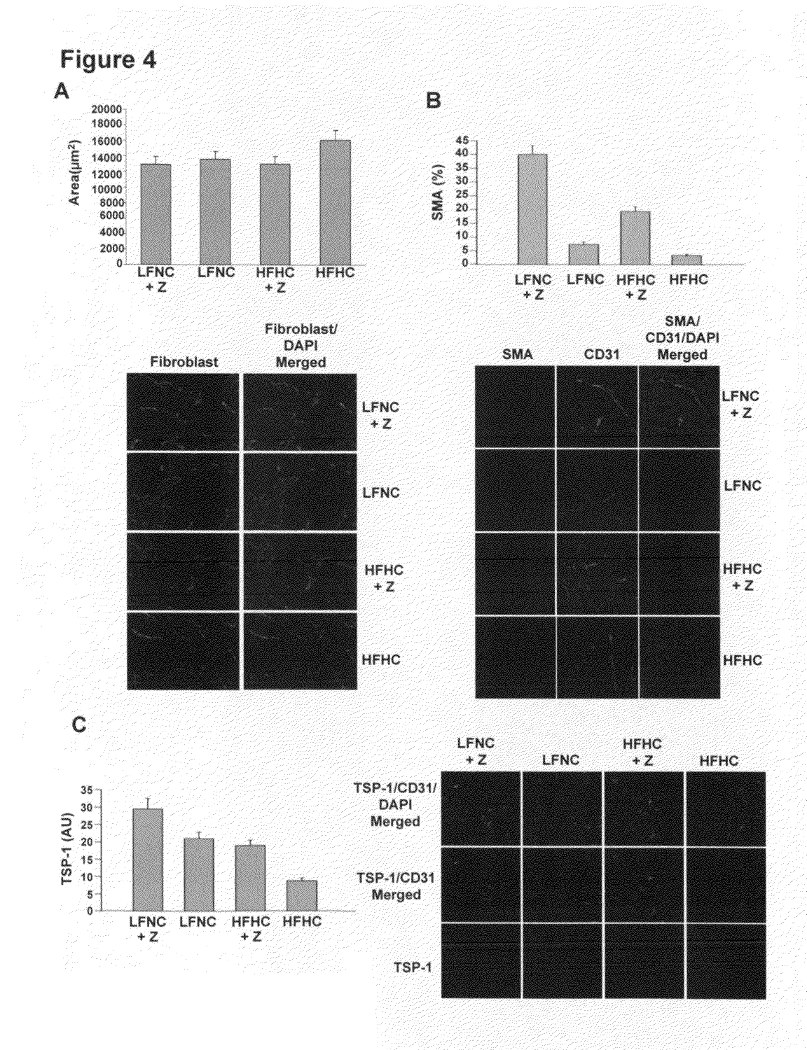

The most dramatic effect elicited by the manipulation of circulating cholesterol levels in the experimental system described herein is its impact on angiogenesis. Reducing cholesterol levels reduces the amount of tumor-associated blood and blood vessels, and increases vessel pericyte coverage (suggesting more stable vascular structure), while raising the tumor-associated levels of thrombospondin-1 (TSP-1), a potent inhibitor of angiogenesis (Example 5 and FIG. 4). These results suggest that a major biological effect of hypercholesterolemia on prostate tumors is increased angiogenesis.

The invention described herein is the administration of an azetidinone drug to reduce angiogenesis. Where the subject is a cancer patient, this drug reduces angiogenesis and ultimately leads to less aggressive tumors.

Ezetimibe is one cholesterol-lowering azetidinone drug that binds to and blocks Niemann-pick C1 Like 1 (NPC1L1), the gut transporter responsible for dietary and biliary cholesterol absorption (Davis, H. R. et al. (2007) J Atheroscler Thromb 14, 99-108; Jurado, J., et al. (2004) Am J Cardiol 93, 641-643; Jurado, J., et al. (2004) Am J Cardiol 93, 641-643; Knopp, R. H., et al. (2003) Eur Heart J 24, 729-741; Davis, H. R., Jr., et al. (2004) J Biol Chem 279, 33586-33592). In the studies described herein using ezetimibe, cholesterol was determined to be responsible for tumor growth, as opposed to other potential tumor growth mediators, such as isoprenoids. The studies described herein, using ezetimibe in combination with other therapeutic and nutritional strategies, support a direct role for circulating cholesterol in the promotion of tumor growth. The findings indicate that an underlying mechanism of this effect occurs at the level of angiogenesis.

The studies indicate that cholesterol levels influenced the extent of angiogenesis in tumors, with the relative density of tumor microvessels corresponding to the level of circulating cholesterol. Consistent with this finding, cholesterol reduction as a result of drug administration increased the intratumoral level of TSP-1, an angiogenesis inhibitor. These studies showed that hypercholesterolemia directly accelerates the growth of prostatic tumors and that pharmacologic reduction in serum cholesterol inhibits tumor angiogenesis to retard cancer growth.

TSP-1 is a multifunctional 450 kDa extracellular matrix glycoprotein and the first endogenous inhibitor of angiogenesis to be discovered. It is critical to the formation and progression of solid tumors including regulating proliferation, adhesion, migration, and angiogenesis. LNCaP cells express TSP-1 as well as its major receptor CD36, and TSP-1 has directly inhibits cell proliferation and stimulates apoptosis.

As demonstrated herein, hypercholesterolemia decreases expression of the angiogenesis inhibitor TSP-1. Thus, it is possible that under hypercholesterolemic conditions the reduction in TSP-1 levels is a direct contributor to the increased angiogenesis, increase in tumor cell proliferation, and decrease in apoptosis observed herein. The potential for cholesterol to regulate TSP-1, and, potentially, other angiogenesis inhibitors, was not previously known.

Angiogenesis is the process by which tissues form new blood vessels from existing ones. It contributes to pathological processes such as tumor growth but also to diseases totally unrelated to cancer. Subjects suffering from a variety of diseases exhibit pathology which are rooted in angiogenic processes that can be treated by administration of azetidinone drugs. Therefore, the invention extends to the administration of the drug to patients with cancer, retinal vascular disease or choroidal vascular disease such as age-related macular degeneration or diabetic retinopathy, rheumatoid arthritis, psoriasis, glaucoma, complications of AIDS or obesity, for example.

The studies described herein demonstrate the surprising result that ezetimibe is an angiogenesis inhibitor. In patients with diseases that are perpetuated by new blood vessel growth, administration of ezetimibe treats the disease.

Circulating cholesterol stimulates the growth of human tumors and, conversely, cholesterol lowering slows tumor growth. As a representative example, we demonstrated this effect on human prostate tumor xenografts in mice. The examples that follow provide experimental evidence that: 1) tumor growth closely corresponded to serum cholesterol level; 2) isocaloric diets eliminated any potential effect of energy imbalance on tumor growth; 3) cholesterol lowering was accomplished with the drug ezetimibe, which blocks both dietary and biliary cholesterol uptake by targeting NPC1L1, a gut transporter thought to be responsible for essentially all uptake of dietary cholesterol; ezetimibe is believed at this time to be specific for NPC1L1; 4) a tumor-promoting effect of hypercholesterolemia was demonstrated in castrated mice, eliminating the possibility that cholesterol exerts an effect on tumor growth via alteration of androgen levels; 5) triglyceride levels were not altered by diet or decreased by ezetimibe treatment, and all liver function tests were normal; and 6) serum cholesterol levels correlated significantly with tumor cholesterol levels, apoptotic tumor cell number, tumor cell proliferation, and MVD.

In the examples below, in the first set of tumor implantation experiments ezetimibe blocked the accelerated tumor growth stimulated by the high fat/high cholesterol (HFHC) diet, and reduced the more modest tumor growth in mice fed a low fat/no cholesterol (LFNC) diet. Ezetimibe works by blocking intestinal uptake of dietary cholesterol and bile-derived cholesterol, thus the drug will reduce circulating cholesterol levels even when there is no cholesterol in the diet. Consistent with this, ezetimibe reduced serum cholesterol levels in mice with no cholesterol in their diet. Unlike statins, which block cholesterol synthesis at an early step in the mevalonate pathway, and thus suppress production of upstream intermediates (including isoprenoids), ezetimibe only blocks cholesterol-uptake and likely has little or no direct effect on other members of the pathway. Ezetimibe has been shown to have a modest effect on reducing triglyceride levels in humans (Jurado, J., Seip, R., and Thompson, P. D. 2004. Effectiveness of ezetimibe in clinical practice. Am J Cardiol 93:641-643; Knopp, R. H., Gitter, H., Truitt, T., Bays, H., Manion, C. V., Lipka, L. J., LeBeaut, A. P., Suresh, R., Yang, B., and Veltri, E. P. 2003. Effects of ezetimibe, a new cholesterol absorption inhibitor, on plasma lipids in patients with primary hypercholesterolemia. Eur Heart J 124:729-741), but in the experiments described herein, no significant reduction of serum triglyceride levels was found, suggesting that altered triglycerides did not contribute to ezetimibe's effects. The experiments demonstrate that a combination of a LFNC diet and ezetimibe reduced tumor growth additively.

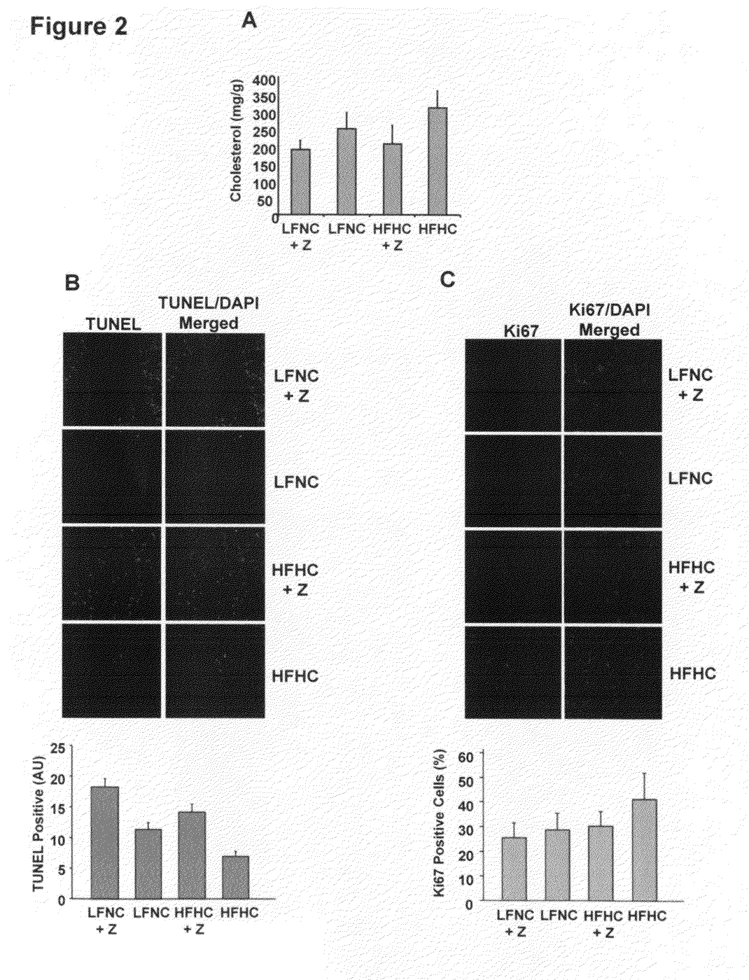

In a separate set of tumor implantation studies, surgically castrated mice were fed isocaloric diets that varied only by cholesterol content. Hormonally intact animals did not demonstrate increased serum cholesterol levels when fed a low fat/high cholesterol (LFHC) diet for ≦90 days, however this diet did raise serum cholesterol levels in castrates. Androgen suppression is known to increase serum cholesterol levels in humans and animals, however the selective effect of dietary cholesterol on circulating cholesterol in the castrate condition was a surprising finding. Preliminary observations indicate that castration plus added dietary cholesterol increases the expression of NPC1L1 in the jejunum, a result that may account for the serum cholesterol elevation. In castrates (Model 2 below), elevated circulating cholesterol was associated with increased tumor take as well as increased tumor growth (FIGS. 2D & E). In this model, cholesterol is the only variable between the two diet groups, and cholesterol has no usable calories, strongly suggesting that elevated serum cholesterol from the diet is responsible for the increased tumor growth in castrated animals.

Androgen-suppressed men lose lean muscle, gain fat, and are at increased risk for cardiovascular disease (Smith, M. R., Finkelstein, J. S., McGovern, F. J., Zietman, A. L., Fallon, M. A., Schoenfeld, D. A., and Kantoff, P. W. 2002. Changes in body composition during androgen deprivation therapy for prostate cancer. J Clin Endocrinol Metab 87:599-603; Foundation, P. C. 2004. Report to the Nation on Prostate Cancer; Smith, J. C., Bennett, S., Evans, L. M., Kynaston, H. G., Parmar, M., Mason, M. D., Cockcroft, J. R., Scanlon, M. F., and Davies, J. S. 2001. The effects of induced hypogonadism on arterial stiffness, body composition, and metabolic parameters in males with prostate cancer. J Clin Endocrinol Metab 86:4261-4267). The experiments herein indicate that hormonal therapy promotes dietary effects on serum cholesterol that do not arise under conditions of normal testicular function, increasing the risk of stimulation of cholesterol-responsive pathways in subclinical tumors. These latter observations suggest that circulating cholesterol may play a role in the development of castration-resistant tumor growth.

Because the experiments described herein involve xenografts generated from tumorigenic cells, the studies address a role for cholesterol in tumor progression, not tumor initiation. However, taken in combination with recent independent, prospective studies showing the protective effect of statin drugs against advanced PCa (Platz, E. A., Leitzmann, M. F., Visvanathan, K., Rimm, E. B., Stampfer, M. J., Willett, W. C., and Giovannucci, E. 2006. Statin drugs and risk of advanced prostate cancer. J Natl Cancer Inst 98:1819-1825; Jacobs, E. J., Rodriguez, C., Bain, E. B., Wang, Y., Thun, M. J., and Calle, E. E. 2007. Cholesterol-lowering drugs and advanced prostate cancer incidence in a large u.s. Cohort. Cancer Epidemiol Biomarkers Prev 16:2213-2217; Murtola, T. J., Tammela, T. L., Lahtela, J., and Auvinen, A. 2007. Cholesterol-Lowering Drugs and Prostate Cancer Risk: A Population-based Case-Control Study. Cancer Epidemiol Biomarkers Prev 16:2226-2232; Flick, E. D., Habel, L. A., Chan, K. A., Van Den Eeden, S. K., Quinn, V. P., Haque, R., Orav, E. J., Seeger, J. D., Sadler, M. C., Quesenberry, C. P., Jr., et al. 2007. Statin Use and Risk of Prostate Cancer in the California Men's Health Study Cohort. Cancer Epidemiol Biomarkers Prev 16:2218-2225), the studies indicated that intervention to inhibit disease progression by lowering cholesterol pharmacologically, possibly in combination with diet, may be effective in some patients.

The invention is further illustrated by the following examples.

### EXAMPLE 1

### Materials and Methods

Antibodies. Anti-CD31 mAb (rat anti-mouse); anti-caveolin pAb (BD Pharmingen. San Jose, Calif.); anti-thrombospondin-1 pAb; anti-caveolin-I pAb; anti-Ki67 pAb (Abcam, Cambridge, Mass.); anti-β actin mAb; anti-smooth muscle actin mAb (Sigma, St Louis, Mo.); anti-phosphotyrosine mAb (Cell Signaling, Danvers, Mass.); Alexa Fluor 488-conjugated goat anti-rat; Alexa Fluor 488-conjugated goat anti-mouse; Alexa Fluor 568-conjugated goat anti-rabbit; Alexa Fluor 568-conjugated goat anti-mouse (Invitrogen, Carlsbad, Calif.); Cy3-conjugated AffiniPure goat anti-rabbit IgG, Fc fragment specific (Jackson ImmunoResearch, West Grove, Pa.).

Mice and Tumor Xenografts. 5 week old SCID mice were obtained from the Massachusetts General Hospital and were fed a low fat/no cholesterol diet (LFNC) (Research Diets, New Brunswick, N.J. diet # D12102) for two weeks, blood was drawn from the saphenous tail vein and the serum cholesterol concentration was determined using the Infinity Cholesterol Liquid Stable Reagent (Thermo Electron Corp.). For intact (non-castrated) mice, the animals were divided into the high fat/high cholesterol diet (HFHC) (Research Diets, diet # D12108) and LFNC groups with and without ezetimibe (30 mg/kg/day; Schering-Plough, New Brunswick, N.J.) and the mice continued on these diets for four weeks. For castrated mice, the mice were divided into 4 groups, 2 groups were castrated, and 2 groups were left intact. The mice were fed either the LFNC, or a low fat/high cholesterol diet (LFHC) (Research Diets, diet #D12104) for 80 days. Xenografts were initiated by injecting LNCaP (2×10⁶ per site) with 1:1 volume of Matrigel (BD BioSciences, San Jose, Calif.) into the 4 dorsal quadrants of each mouse. In order to eliminate any injection bias, the mice were randomized prior to implantation and the implanter was blinded to which group each mouse was assigned. All animal procedures were done in compliance with Children's Hospital Boston's animal care and use policies. Tumors were measured daily from the initiation of the first palpable tumors and the mice sacrificed prior to reaching the maximum tumor burden (13-23 days post implantation). Terminal bleeds were taken (Cardiac puncture) for serology (triglyceride, bilirubin and other liver function tests were performed in the Dept. of Laboratory Medicine, Children's Hospital Boston, androgen levels were determined by a testosterone EIA, Diagnostic Systems Laboratories, Webster, Tex.). Tumors were removed, measured, weighed and either placed in OCT solution (Tissue-Tek, Torrance, Calif.) or snap frozen.

Cell culture. LNCaP human prostate tumor cells (American Type Culture Collection, Manassas, Va.), which do not express either caveolin (Zhuang, L., Lin, J., Lu, M. L., Solomon, K. R., and Freeman, M. R. 2002. Cholesterol-rich lipid rafts mediate akt-regulated survival in prostate cancer cells. Cancer Res 62:2227-2231) or PTEN (Wu, X., Senechal, K., Neshat, M. S., Whang, Y. E., and Sawyers, C. L. 1998. The PTEN/MMAC1 tumor suppressor phosphatase functions as a negative regulator of the phosphoinositide 3-kinase/Akt pathway. Proc Natl Acad Sci U.S.A 95:15587-15591) were cultured in RPMI (Invitrogen, Carlsbad, Calif.) media supplemented with 10% FBS and 1% Penicillin/Streptomycin at 5% CO₂ at 37° C.

Tumor cholesterol analysis. Tumors were finely minced in phosphate buffered saline (PBS) on ice and the level of cholesterol was determined as described previously (Boucher, K., Siegel, C. S., Sharma, P., Hauschka, P. V., and Solomon, K. R. 2006. HMG-CoA reductase inhibitors induce apoptosis in pericytes. Microvasc Res 71:91-102).

Apoptosis. Percentage of apoptotic cells in tumor sections was determined by TUNEL assay using the In Situ Cell Death Detection Kit (Roche Diagnostics Corp. Indianapolis, Ind.). Briefly, frozen tumor sections were fixed in 4% paraformaldehyde (PFA), permeabilized and the DNA stained with fluorescein following the manufacturer's instructions. Nuclei were counterstained with DAPI (Vector Labs, Burlingame, Calif.). Images were captured using a Zeiss microscope and the positive cells and nuclei were counted using Axiovision software 4.0.

Immunofluorescence. Tumor samples frozen in OCT were sectioned (3-20 μm thick), mounted on Superfrost slides (ThermoFisher Scientific, Waltham, Mass.), and air-dried for 30 min. Sections were then fixed using cold acetone (5 min), followed by 1:1 acetone:chloroform (5 min), and then acetone (5 min) or by using 4% PFA at room temp (30 min) followed by 0.025% PBS/Triton X-100 (5 min) to permeabilize the cells. Sections were washed with cold PBS 3× for 5 min each and were incubated in a protein blocking solution of PBS/Tween (0.1%) with 5% bovine serum albumin (BSA; Sigma) at room temp (30 min) and were then washed in cold PBS 3× for 5 min each. The appropriate primary antibodies diluted 1:200-1:1000 in blocking solution were incubated with the sections overnight at 4° C. Sections were washed 3× for 5 min in cold PBS, and incubated in blocking solution (10 min). The sections were then incubated with the appropriate fluorescent secondary reporter antibodies diluted 1:500-1:5000 in blocking solution at room temp (30 min) followed by washing 3× in PBS. Nuclei were counter-stained with DAPI. Double staining of CD31 with TSP-1 and CD31 with SMA were performed sequentially as described above. Blood vessel analysis was done using IPLab software (BD Biosciences Bioimaging, Rockville, Md.) as previously described (Bielenberg, D. R., Hida, Y., Shimizu, A., Kaipainen, A., Kreuter, M., Kim, C. C., and Klagsbrun, M. 2004. Semaphorin 3F, a chemorepulsant for endothelial cells, induces a poorly vascularized, encapsulated, nonmetastatic tumor phenotype. J Clin Invest 114:1260-1271). Thombospndin-1 (TSP-1) analysis was done using the outline spline in AxioVision 4.0 software. Ki67 positive cells and nuclei were quantified using AxioVision 4.0 software.

Lysates and Immunoblotting. Tumors were finely minced in PBS on ice and lipid raft and non-raft fractions were prepared as previously described (Solomon, K. R., Danciu, T. E., Adolphson, L. D., Hecht, L. E., and Hauschka, P. V. 2000. Caveolin-enriched membrane signaling complexes in human and murine osteoblasts. J Bone Miner Res 15:2380-2390; Solomon, K. R., Mallory, M. A., and Finberg, R. W. 1998. Determination of the non-ionic detergent insolubility and phosphoprotein associations of glycosylphosphatidylinositol-anchored proteins expressed on T cells. Biochem J 334:325-333). Protein concentrations were determined by microBCA (Pierce/Thermo Scientific) and equal amounts of the lysates were subjected to SDS-PAGE and immunoblotting as described previously (Zhuang, L., Kim, J., Adam, R. M.-, Solomon, K. R., and Freeman, M. R. 2005. Cholesterol targeting alters lipid raft composition and cell survival in prostate cancer cells and xenografts. J Clin Invest 115:959-968).

Hemoglobin Assay. Tumors were finely minced in 1 ml of fresh PBS on ice. The minced tissue suspensions were centrifuged at 10,000 g at 4° C. (2 min) and the supernatant was removed. The optical density (OD) of the clarified supernatants was read at 650 nm (background) and 530 nm (hemoglobin) using a spectrophotometer (Boyle, M. D., and Ohanian, S. H. 1980. Evidence for the influence of the initial complement components on the assembly and activity of the membrane attack complex. J Immunol 124:2824-2827; Gee, A. P., Boyle, M. D., and Borsos, T. 1980. Distinction between C8-mediated and C8/C9-mediated hemolysis on the basis of independent 86Rb and hemoglobin release. J Immunol 124:1905-1910).

Statistics. To assess the effects of drug and diet on growth as well as microvessel density, pericyte coverage, and TSP-1 levels in xenograft tumors in SCID mice, a mixed-model analysis of variance (ANOVA) was used in order to account for the repeated measurements within the same tumor or animal and multiple time points (Laird, N. M., and Ware, J. H. 1982. Random-effects models for longitudinal data. Biometrics 38:963-974.). Compound symmetry covariance structure was incorporated to model the within-animal correlation and provided the best fit to the data as judged by Akaike's information criterion (AIC) (Akaike, H. 1981. Likelihood of a model and information criteria. Journal of Econometrics 16:3-14). Logistic regression analysis was applied to evaluate whether the proportion of tumor take was significantly different between two diets (LFNC vs. LFHC) used in castrated animals at the various time points. Here, the binary endpoint was defined as the presence or absence of a palpable tumor and the likelihood ratio test (LRT) was used as the criterion for significance (Hosmer, D., and Lemeshow, S. 2000. Applied logistic regression. In Applied logistic regression. New York: John Wiley & Sons. 143-202). Data for tumor cholesterol, apoptosis, and proliferation were analyzed using two-way ANOVA with diet and drug as factors in the 2×2 factorial experiment and diet-by-drug interaction term in the models to ascertain whether the diet and drug effects are independent main effects or conditional (Montgomery, D. 2001. Design and analysis of experiments. New York: John Wiley & Sons. 170-217). Simple comparisons were performed using the standard unpaired Student's t test. Statistical analysis was performed with SPSS software (version 15.0, SPSS Inc., Chicago, Ill.). Two-tailed values of p<0.05 were considered statistically significant.

### EXAMPLE 2

### Effect of Diet Alone on Serum Cholesterol

The atherogenic Paigen diet is the standard method for raising levels of circulating cholesterol in mice. However, the Paigen diet causes severe liver toxicity and contains sodium cholate, a bile acid and liver toxin. Therefore, the mice were fed a low fat/high cholesterol (LFHC) diet without sodium cholate. This allowed for the isolation of the effect of cholesterol from other factors under conditions more relevant to human diets.

Hormonally intact (uncastrated) SCID mice did not exhibit a significant rise in serum cholesterol levels on the LFHC diet (135±15.2 mg/dL before vs. 144 ±50.8 mg/dL after diet for 73 days (d) (n=10, p=0.57)), whereas castrated mice exhibited a significant increase in serum cholesterol on the same diet (140±9.64 before vs. 176±22.5 mg/dL after diet for 73 d (n=10, p=0.0002)). This effect is attributed to dietary cholesterol because a low fat/no cholesterol (LFNC) diet did not raise circulating cholesterol levels (138±9.44 mg/dL before vs. 146±17.6 mg/dL after LFNC diet for 73 d (n=10, p=0.23)). Consistent with these findings are reports that androgen-deprived humans and rodents have elevated serum cholesterol (Fillios, L. C. 1957. The gonadal regulation of cholesteremia in the rat. Endocrinology 60:22-27; Haug, A., Hostmark, A. T., and Spydevold, O. 1984. Plasma lipoprotein responses to castration and androgen substitution in rats. Metabolism 33:465-470; Haug, A., Hostmark, A. T., Spydevold, O., and Eilertsen, E. 1986. Hypercholesterolaemia, hypotriacylglycerolaemia and increased lipoprotein lipase activity following orchidectomy in rats. Acta Endocrinol (Copenh) 113:133-139; Leblanc, M., Belanger, M. C., Julien, P., Tchernof, A., Labrie, C., Belanger, A., and Labrie, F. 2004. Plasma lipoprotein profile in the male cynomolgus monkey under normal, hypogonadal, and combined androgen blockade conditions. J Clin Endocrinol Metab 89:1849-1857; Nishiyama, T., Ishizaki, F., Anraku, T., Shimura, H., and Takahashi, K. 2005. The influence of androgen deprivation therapy on metabolism in patients with prostate cancer. J Clin Endocrinol Metab 90:657-660; Pick, R., Stamler, J., Rodbard, S., and Katz, L. N. 1959. Effects of testosterone and castration on cholesteremia and atherogenesis in chicks on high fat, high cholesterol diets. Circ Res 7:202-204; Smith, M. R., Finkelstein, J. S., McGovern, F. J., Zietman, A. L., Fallon, M. A., Schoenfeld, D. A., and Kantoff, P. W. 2002. Changes in body composition during androgen deprivation therapy for prostate cancer. J Clin Endocrinol Metab 87:599-603; Yannucci, J., Manola, J., Garnick, M. B., Bhat, G., and Bubley, G. J. 2006. The effect of androgen deprivation therapy on fasting serum lipid and glucose parameters. J Urol 176:520-525).

### EXAMPLE 3

### Effect of Diet and Ezetimibe on Serum Cholesterol

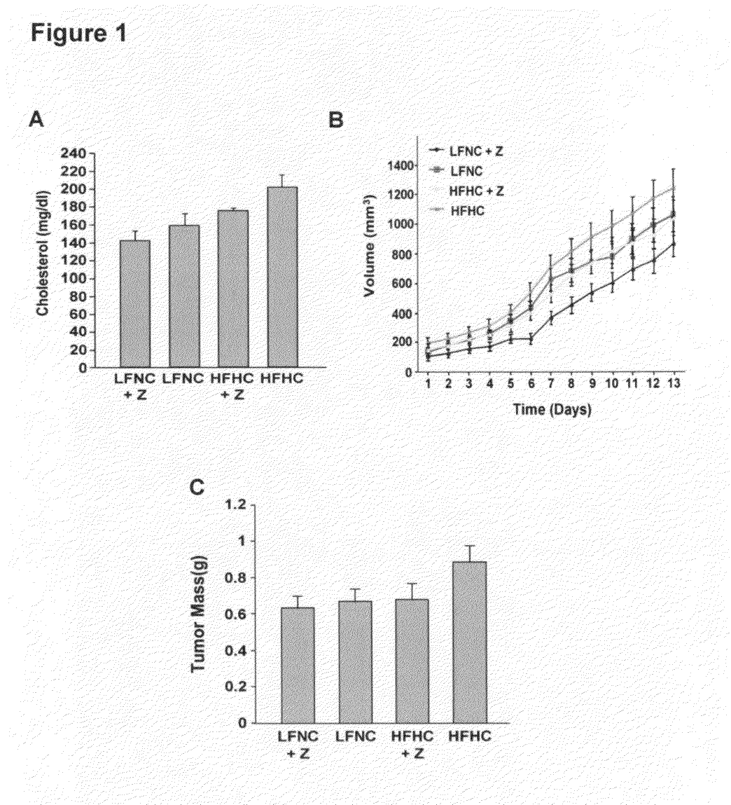

Given the above results, two independent approaches were taken to specifically alter cholesterol levels in vivo (FIG. 1). In Model 1 circulating cholesterol was raised with a high fat/high cholesterol (HFHC) diet, which raised cholesterol in intact animals (FIG. 2A), as compared with mice fed a LFNC diet. These diets are isocaloric, excluding any possibility of an energy effect. A subset of mice on both diets were treated with ezetimibe (30 mg/kg/d) to lower cholesterol. In Model 2 circulating cholesterol was raised in castrated mice through the use of the LFHC diet and these animals were compared to mice on an isocaloric LFNC diet.

Animals were given various ezetimibe (Z)-diet combinations (see FIG. 2) for 4 weeks after which the mice were bled by small tail vein incision, and cholesterol measured in the collected serum via Infinity colorimetric assay. Initially, cholesterol levels were normalized in all animals using the LFNC diet for 2 weeks (w). Average cholesterol level was 147.47±17.25 mg/dL (range 112.07-214.66 mg/dL). Two mice with the lowest and highest cholesterol levels were eliminated from the cohort of mice because cholesterol varied by more than two standard deviations from the average. Fifty-eight mice were then randomly assigned to 1 of 4 diet/drug groups: (1) LFNC, (2) LFNC+ezetimibe (Z), (3) HFHC, and (4) HFHC+Z. Calorie intake was set at 21.18 Kcal/d in all groups. Observation indicated that ezetimibe did not alter feeding behavior. Animals were kept on the regimens for 4 weeks, and cholesterol levels determined every other week. At 4 weeks the serum cholesterol levels were significantly different between the four cohorts (FIG. 2A). Ezetimibe caused significant reductions in serum cholesterol in both the LFNC and HFHC diet groups.

### EXAMPLE 4

### Effect of Diet and Ezetimibe on Tumor Xenograft

Following alteration of cholesterol levels, the mice from Example 3 were injected subcutaneously with 2×10⁶ LNCaP cells in their flanks (4 tumors/mouse). Regimens were continued following implantation, and the experiment continued for 13 days following the initial appearance of tumors. Tumor volume was measured daily (FIG. 2B). Significantly reduced tumor growth rates were observed with the LFNC diet (p=0.048) and by added ezetimibe (p=0.035). The overall effect of diet and ezetimibe was independent and additive, not synergistic. HFHC tumor wet weights at sacrifice were significantly larger than tumors in the other groups (FIG. 2C; LFNC drug (0.63±0.55 g) vs. HFHC (0.88±0.71 g), p=0.021; LFNC (0.67±0.56 g) vs. HFHC, p=0.037). Tumor take was >95% in all groups and no significant differences in tumor take were observed. The HFHC diet did not result in statistically significant increases in serum triglycerides, nor were triglyceride levels significantly reduced in the ezetimibe cohorts (data not shown). Serological testing (AST, ALP, bilirubin etc) indicated no liver dysfunction in any mouse (data not shown) and diet did not affect testosterone levels.

In Model 2, above, diets that differed only in the amounts of cholesterol were used, because increased dietary cholesterol was sufficient to raise circulating cholesterol in castrated SCID mice. Because LNCaP cells are androgen-dependent and will not grow (or grow poorly) in castrated mice, the LNCaP cells used here were engineered to produce soluble heparin-binding EGF-like growth factor (sHB/LNCaP cells), a prostate stroma-derived growth factor. These cells form tumors in castrated mice. Castration reduced the level of circulating testosterone from 10.41±5.27 to 0.29±0.05 ng/ml. Tumor take was significantly greater in the hypercholesterolemic, LFHC mice in comparison to the normocholesterolemic, LFNC mice (FIG. 2D). Tumor volume was also greater in the LFHC group (FIG. 2E), but these data were not statistically significant due to the large variation in tumor take (too few tumors grew in the LFNC mice). Overall, these data indicated that the effect of cholesterol on the growth of implanted tumors was independent of the animal's androgen status.

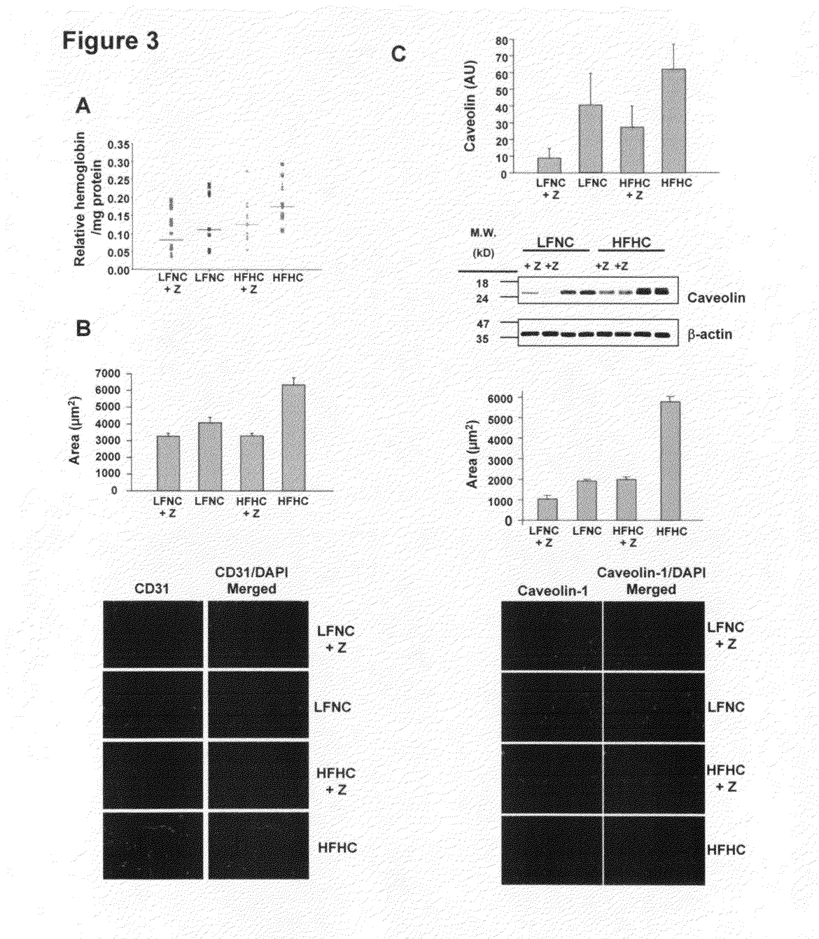

In order to investigate the mechanisms behind the apparent tumor-promoting effect of elevated cholesterol, the tumors were examined using a variety of approaches. The level of membrane cholesterol in the tumors reflected the serum cholesterol levels in vivo (FIG. 3A). Tumor cholesterol levels were independently affected by both ezetimibe and by diet. Apoptosis and cell proliferation were evaluated quantitatively in the Model 1 tumors. Apoptosis was significantly increased by the LFNC diet (p21 0.0001) and independently by ezetimibe (p<0.0001), however, no synergy was observed (p=0.85) (FIG. 3B). Cell proliferation, as measured by Ki67 staining, was greater in the HFHC tumors and, independently, when ezetimibe was omitted (FIG. 3C). A significant diet/ezetimibe interaction (p=0.027) was observed using cell proliferation as an endpoint.

### EXAMPLE 5

### Effect of Diet and Ezetimibe on Factors Associated with Angiogenesis

In processing the above xenografts, tumors from the HFHC mice were bloodier than tumors from other cohorts. Relative hemoglobin levels correlated positively with circulating cholesterol level (FIG. 4A), suggesting a potential effect of cholesterol on vascular penetration into the tumors. Microvessel density (MVD) was quantified using an antibody for CD31 (PECAM), an endothelial cell marker. MVD was significantly suppressed by both ezetimibe and by the LFNC diet when compared to the HFHC diet (FIG. 4B). A similar result was observed when caveolin-1, which is expressed at high levels by murine endothelial cells but not by LNCaP cells, was used as an independent assessment of MVD (Zhuang, L., Lin, J., Lu, M. L., Solomon, K. R., and Freeman, M. R. 2002. Cholesterol-rich lipid rafts mediate akt-regulated survival in prostate cancer cells. Cancer Res 62:2227-2231; Gratton, J. P., Bernatchez, P., and Sessa, W. C. 2004. Caveolae and caveolins in the cardiovascular system. Circ Res 94:1408-14.17; Lu, M. L., Schneider, M. C., Zheng, Y., Zhang, X., and Richie, J. P. 2001. Caveolin-1 interacts with androgen receptor. A positive modulator of androgen receptor mediated transactivation. J Biol Chem 276:13442-13451) (FIGS. 4C, D). MVD was not simply a reflection of tumor size because no correlation was observed between tumor size and MVD when similar-sized tumors from each cohort were compared (r=0.05; p=0.39).

Blood vessels undergoing rapid angiogenesis in tumors tend to exhibit poor vascular morphology, characterized by low pericyte recruitment (Bergers, G., and Song, S. 2005. The role of pericytes in blood-vessel formation and maintenance. Neuro Oncol 7:452-464). Pericyte coverage of vessels was dramatically increased in the ezetimibe groups (FIG. 4D), suggesting a strong de-stabilizing effect of circulating cholesterol on blood vessel structure. These results strongly suggest that one or more angiogenic factor(s) might be responsible for the observed effects, but after extensive testing, no differences were found in the cohorts in the pro-angiogenic factors VEGF or bFGF expression (data not shown). Hypercholesterolemia leads to increased activation of the serine-threonine kinase Akt (Zhuang, L., Kim, J., Adam, R. M., Solomon, K. R., and Freeman, M. R. 2005. Cholesterol targeting alters lipid raft composition and cell survival in prostate cancer cells and xenografts. J Clin Invest 115:959-968.). Akt reduces expression of TSP-1, a potent angiogenic suppressor (Niu, Q., Perruzzi, C., Voskas, D., Lawler, J., Dumont, D. J., and Benjamin, L. E. 2004. Inhibition of Tie-2 signaling induces endothelial cell apoptosis, decreases Akt signaling, and induces endothelial cell expression of the endogenous anti-angiogenic molecule, thrombospondin-1. Cancer Biol Ther 3:402-405). There were highly significant differences in thrombospondin-1 (TSP-1) levels, which were enhanced by both the LFNC diet and ezetimibe (FIG. 4E). These results indicated a pronounced effect of circulating cholesterol on mechanisms of angiogenesis.

### EXAMPLE 6

### Ezetimibe Administration as Treatment for Ocular Angiogenesis

The usefulness of azetidinones for treating non-tumor disease are shown using azetidinone administration with a murine model of human ocular angiogenesis. As representative of the class of azetidinones, ezetimibe is administered. A power analysis determines the sufficient numbers of mice to be randomized to each group (Ezetimibe treated and control). To detect a 40% difference in angiogenesis between the control and Ezetimibe treated groups, a sample size of 39 animals provides 90% statistical power (two-tailed α=0.05, β=0.10) to detect a minimum difference of 40% in angiogenesis between the two groups assuming a variability of 50% using the unpaired Student's t-test (version 5.0, nQuery Advisor, Statistical Solutions, Boston, Mass.). To conduct the experiment and to account for unexpected or early animal death (2 mice), 40+1=41 animals are randomly assigned to each group. Analysis of the data is performed using the SPSS statistical package (version 13.0, SPSS Inc., Chicago, Ill.).

Choroidal neovascularization (CNV) is induced in 5-6-week-old C57BL/6 mice by laser photocoagulation-induced rupture of Bruch's membrane (Tobe, T et al., (1998) Am J Pathol. 153(5):1641-6). Mice are anesthetized with ketamine/xylazine and pupils are dilated with 1% tropicamide. Three bums of 532 nm diode laser photocoagulation (75 mm spot size, 120 mW, 0.1 sec duration) are delivered to retinas (9, 12, and 3 o'clock positions of the posterior pole) using a slit lamp mounted OcuLight GL diode laser (Iridex, Mountain View, Calif.) with a handheld cover slip as a contact lens to view the retina. Only burns in which a bubble is produced (indicating Bruch's membrane rupture) are included in the study.

For 2 months following laser treatment, mice are treated with or without ezetimibe (30-60 mg/kg/day mixed into the food). After 2 months, mice are perfused with 1 ml of PBS containing 50 mg/ml of fluorescein-labeled dextran (2×10⁶ average molecular weight, Sigma-Aldrich, St. Louis, Mo.), eyes are removed and fixed for 1 h in 10% phosphate-buffered formalin. After dissecting free the cornea, lens, and retina, four radial cuts are made in the eyecup allowing it to be flat mounted in aqueous mounting medium. Flat mounts are then examined by fluorescence microscopy and images are digitized using a three-color CCD video camera and a frame grabber. Image analysis software (Image-Pro Plus, Media Cybernetics, Silver Spring, Md.) is then used to measure the total area of CNV at each rupture in an analysis blinded as to study treatments and groups.

While the invention has been described in connection with specific embodiments, it will be understood that it is capable of further modifications. Therefore, this application is intended to cover any variations, uses, or adaptations of the invention that follow in general, the principles of the invention, including departures from the present disclosure that come within known or customary practice within the art. Other embodiments are within the claims.

All publications, patent applications and patents mentioned in this specification are herein incorporated by reference.

What is claimed is:

1. A method of inhibiting angiogenesis in a subject with an angiogenesis-related pathology comprising administering an azetidinone to the subject.

2. The method of claim 1 wherein the condition is selected from the group consisting of macular degeneration, rheumatoid arthritis, psoriasis, diabetes, glaucoma and obesity.

3. The method of claim 1 wherein the inhibition of angiogenesis is attained by administering a therapeutic amount of ezetimibe.

4. The method of claim 1 wherein the inhibition of angiogenesis is attained by administering ezetimibe with an angiogenesis inhibitor that includes angiostatin, endostatin, TNP-470, thalidomide, aptamer antagonist of VEGF, batimastat, captopril, interleukin 12, lavendustin A, medroxypregesterone acetate, recombinant human platelet factor 4(rPF4), taxol, tecogalan(=SP-PG, DS-4152), thrombospondin, TNP-470 (=AGM-1470) and bevacizumab (Avastin®).

5. The method of claim 1 wherein the azetidinone is ezetimibe and further comprising maintaining a low fat/low cholesterol diet regimen.

6. A method of inhibiting angiogenesis in a solid tumor comprising administering to a subject with a solid tumor an azetidinone and an angiogenesis inhibitor compound.

7. The method of claim 6 wherein the azetidinone is ezetimibe in an amount that is inhibitory for angiogenesis.

8. The method of claim 6 wherein the angiogenesis inhibitor compound is selected from the group consisting of angiostatin, endostatin, TNP-470, thalidomide, aptamer antagonist of VEGF, batimastat, captopril, interleukin 12, lavendustin A, medroxypregesterone acetate, recombinant human platelet factor 4(rPF4), taxol, tecogalan(=SP-PG, DS-4152), thrombospondin, TNP-470 (=AGM-1470) and bevacizumab (Avastin®).

9. The method of claim 6, further comprising maintaining a low fat/low cholesterol diet regimen.

10. The method of claim 6 wherein the solid tumor is located in the prostate, breast, pancreas, liver, brain, lung, kidney, bladder, bone, heart, testis, uterus, ovaries, neck, mouth, nose, eye, head, colon, rectum; stomach, muscle, cartilage, skin or esophagus.

11. A method of inhibiting tumor cell proliferation comprising administering a therapeutic amount of an azetidinone and a therapeutic amount of an angiogenesis inhibitor to a subject with a solid tumor.

12. The method of claim 11 wherein the solid tumor is located in the prostate, breast, pancreas, liver, brain, lung, kidney, bladder,. bone, heart, testis, uterus, ovaries, neck, mouth, nose, eye, head, colon, rectum, stomach, muscle, cartilage, skin or esophagus.

13. The method of claim 11 wherein the azetidinone is ezetimibe and the angiogenesis inhibitor is selected from the group consisting of angiostatin, endostatin, TNP-470, thalidomide, aptamer antagonist of VEGF, batimastat, captopril, interleukin 12, lavendustin A, medroxypregesterone acetate, recombinant human platelet factor 4(rPF4), taxol, tecogalan(=SP-PG, DS-4152), thrombospondin, TNP-470 (=AGM-1470) and bevacizumab (Avastin®).

14. The method of claim 11 further comprising administering a therapeutic amount of an anticancer agent selected from the group consisting of a steroidal antiandrogen, a non steroidal antiandrogen, an estrogen, diethylstilbestrol, a conjugated estrogen, a selective estrogen receptor modulator (SERM), a taxane, goserelin acetate (ZOLADEX®), and leuprolide acetate (LUPRON®).

15. A method of inhibiting prostate tumor growth without reducing testosterone levels comprising administering a therapeutic amount of an azetidinone.

16. The method of claim 15 wherein the azetidinone is ezetimibe.

17. The method of claim 15 further comprising administering a therapeutic amount of another angiogenesis inhibitor.

18. The method of claim 17 wherein the angiogenesis inhibitor is selected from the group consisting of angiostatin, endostatin. TNP-470, thalidomide, aptamer antagonist of VEGF, batimastat, captopril, interleukin 12, lavendustin A, medroxypregesterone acetate, recombinant human platelet factor 4 (rPF4), taxol, tecogalan (=SP-PG (Sulfated polysaccharide-peptidoglycan), DS-4152), thrombospondin, TNP-470 (=AGM-1470)(the fumagillin analog TNP-470) and bevacizumab (Avastin®).

19. The method of claim 15 further comprising administering a therapeutic amount of a chemotherapeutic agent.

20. A method of inhibiting prostate tumor cell proliferation in androgen-suppressed males comprising administering a therapeutic amount of an azetidinone.

21. The method of claim 20 wherein the azetidinone is ezetimibe.

22. The method of claim 20 wherein the inhibition is attained by administering a therapeutic amount of ezetimibe with a therapeutic amount of an angiogenesis inhibitor class of compounds that includes angiostatin, endostatin, TNP-470, thalidomide, aptamer antagonist. of VEGF, batimastat, captopril, interleukin 12, lavendustin A, medroxypregesterone acetate, recombinant human platelet factor 4(rPF4), taxol, tecogalan(=SP-PG, DS-4152), thrombospondin, TNP-470 (=AGM-1470) and bevacizumab (Avastin®).

23. The method of claim 20 wherein the inhibition is attained by administering a therapeutic amount of ezetimibe with a therapeutic amount of a chemotherapeutic agent.
